(19) United States
(12) Patent Application Publication
(10) Pub. No.: US 2025/0114580 A1
(43) Pub. Date: Apr. 10, 2025

(54) Needle Arrays, Wearable Medicament Delivery Devices, and Methods of Medicament Delivery Utilizing Sensing Microneedles

(71) Applicant: University College Cork - National University of Ireland, Cork, Cork (IE)

(72) Inventors: Alexander LYNESS, West Chester, PA (US); Conor O'MAHONY, Belgooly (IE); Carlo WEBSTER, Cork (IE); Bingxi YAN, Downingtown, PA (US); Len T. MAGARA, Portsmouth, NH (US); Andrea BOCCHINO, Cork (IE); Ryan Thomas SEBASTIAN, Bandon (IE); Fjodors TJULKINS, Riga (LV)

(21) Appl. No.: 18/906,014

(22) Filed: Oct. 3, 2024

Related U.S. Application Data
(60) Provisional application No. 63/542,655, filed on Oct. 5, 2023.

(51) Int. Cl. A61M 37/00

(52) U.S. Cl. CPC A61M 37/0015; A61M 2037/0023; A61M 2037/003; A61M 2202/0007; A61M 2205/0244; A61M 2205/10; A61M 2205/123; A61M 2205/3327; A61M 2205/502; A61M 2209/088; A61M 2210/04

(57) ABSTRACT

This disclosure is directed to needle arrays, wearable medicament delivery devices, and targeted methods of medicament delivery into the skin utilizing sensing microneedles. The needle array includes an injection needle that can deliver the medicament to a patient and a sensing microneedle that can sense a characteristic. The characteristic can be associated with a depth that the sensing microneedle is inserted into the patient. A tip of the sensing microneedle is offset from a tip of the injection needle.

### CROSS-REFERENCE TO RELATED APPLICATION

The present application claims priority to U.S. Provisional Patent Application No. 63/542,655, filed on Oct. 5, 2023, the entire disclosure of which is incorporated herein by reference.

### TECHNICAL FIELD

This disclosure is generally directed to the field of microneedles, and more particularly to needle arrays, wearable medicament delivery devices, and methods of medicament delivery utilizing sensing microneedles that sense characteristics such as injection depth.

### BACKGROUND

Some medicament delivery devices can be worn by the patient. Such wearable medicament delivery devices can be battery-powered and can include a pre-fillable cartridge filled with the medicament. The wearable medicament delivery devices can be loaded with the medicament-filled container and self-administered by the patient. The wearable medicament delivery devices can deliver the medicament subcutaneously into the patient. The wearable medicament delivery devices can include visual or audio feedback to inform the patient of the status of the injection. Some of the wearable medicament delivery devices can be for single use and can be intended exclusively for use with co-packaged prefilled medicament containers for delivering a fixed dose of the prefilled medicament into the patient in a predetermined amount of time controlled by the wearable medicament delivery device.

### SUMMARY

The inventors recognized several shortcomings with existing wearable medicament delivery devices. For example, there exists a need for wearable medicament delivery devices that can accurately sense a characteristic associated with the wearable medicament delivery device for determining whether the wearable medicament delivery device is properly attached to the patient before the medicament is injected and/or during injection of the medicament. The inventors also recognized that sensing the characteristic associated with the wearable medicament delivery device should be achieved in a manner that is minimally invasive and does not introduce any pain to the patient. The inventors also recognized the need to reduce instances of wet injections (i.e., incomplete injections that cause the medicament to leak onto an external skin surface) and/or report on other anomalies, such as adverse events, that may occur during an injection.

These needs are met, to a great extent, by a needle array for delivering a medicament according to one general aspect of the invention. The needle array includes an injection needle configured to deliver the medicament to a patient. The array also includes a sensing microneedle configured to sense a characteristic associated with contact between the sensing microneedle and the patient. A tip of the sensing microneedle is offset from a tip of the injection needle.

Implementations may include one or more of the following features. The needle array where the characteristic associated with the contact between the sensing microneedle and the patient is associated with a depth of penetration of the sensing microneedle into the patient. The depth of penetration is quantified by an amount of contact between the sensing microneedle and the patient along a length of the sensing microneedle. The length being measured between a base of the needle array and the tip of the sensing microneedle. The injection needle and the sensing microneedle are each immovably fixed to a base of the needle array, and the tip of the injection needle extends distally to or beyond the tip of the sensing microneedle. The sensing microneedle is a solid microneedle and the injection needle is a hollow needle. The hollow needle is a microneedle, and the sensing microneedle and the microneedle each have a length of less than 1 mm. The hollow needle is a cannula needle having a length of 1 mm or greater. When the needle array is at a target position on the patient, the tip of the sensing microneedle extends at least through a stratum corneum layer of the patient. When the needle array is at a target position on the patient, the tip of sensing microneedle is at least 10 μm from an outer surface of skin of the patient. The tip of the sensing microneedle extends less than 1000 μm from a base of the needle array. The tip of the sensing microneedle extends less than 500 μm from the base of the needle array. The characteristic is at least one of impedance, resistance, or capacitance. The sensing microneedle is one of a plurality of sensing microneedles configured to sense the characteristic. The plurality of sensing microneedles are arranged in a first sensing microneedle sub-array and a second microneedle sub-array, and the injection needle is between the first sensing microneedle sub-array and the second microneedle sub-array. The injection needle is a first injection needle and the needle array further may include a second injection needle configured to also deliver the medicament to the patient, and the first injection needle and the second injection needle are each hollow microneedles. The characteristic changes in response to a depth that the sensing microneedle is inserted into the patient.

Another general aspect is directed to a wearable medicament delivery device that includes a container containing a medicament. The wearable medicament delivery device also includes a needle array. The needle array may include an injection needle configured to deliver the medicament to a patient, and a sensing microneedle configured to sense a characteristic associated with a depth that the sensing microneedle is inserted into the patient. A tip of the sensing microneedle is offset from a tip of the injection needle. The wearable medicament delivery device also includes a pump operatively connected to the injection needle and to the container. The wearable medicament delivery device also includes a controller operatively connected to the pump and the sensing microneedle. The controller being configured to prevent operation of the pump when the characteristic sensed by the sensing microneedle is outside a target range.

Implementations may include one or more of the following features. The controller is configured to operate the pump when the characteristic sensed by the sensing microneedle is within the target range. The characteristic is impedance. The first housing is reusable and houses the controller and the second housing is disposable and houses the pump, the container, and the needle array. The wearable medicament delivery device may include an adhesive patch attached to the second housing and configured to adhere the wearable medicament delivery device to the patient. The container contains more than 0.5 ml of the medicament and the controller is configured to control the pump to deliver an entirety of the medicament to the patient via the injection needle.

Another general aspect includes a method of delivering a medicament. The method includes sensing, using a sensing microneedle, a characteristic associated with a depth that the sensing microneedle is inserted into a patient. The method also includes determining, using a controller operatively connected to the sensing microneedle, that the characteristic is within a target range. The method also includes switching a state of the controller in response to determining that the characteristic is within the target range from a default state to a ready-to-inject state. In the default state, the controller does not operate a pump in response to receiving instructions from a user interface to deliver the medicament. In the ready-to-inject state, the controller operates the pump in response to receiving instructions from the user interface to deliver the medicament.

Implementations may include one or more of the following features. The method may include: determining, using the controller, that an injection has started; sensing, using the sensing microneedle and during the injection, a second characteristic; determining, using the controller, that the second characteristic is outside the target range; and switching, in response to determining that the second characteristic is outside the target range, the state of the controller from the ready-to-inject state to the default state. The method may include: sensing, using the sensing microneedle and after switching the controller to the default state, a third characteristic; determining, using the controller, that the third characteristic is within the target range; and switching, in response to determining that the third characteristic is within the target range, the state of the controller from the default state to the ready-to-inject state. The method may include: determining, using the controller, that injection of the medicament has been completed; and switching, in response to determining that the injection of the medicament has been completed, the state of the controller from the ready-to-inject state to a spent state, in which the controller does not operate the pump in response to receiving instructions from the user interface to deliver the medicament independent from the sensing microneedle.

Various additional features and advantages of this invention will become apparent to those of ordinary skill in the art upon review of the following detailed description of the illustrative embodiments taken in conjunction with the accompanying drawings.

### BRIEF DESCRIPTION OF THE DRAWINGS

The following detailed description is better understood when read in conjunction with the appended drawings. For the purposes of illustration, examples are shown in the drawings; however, the subject matter is not limited to the specific elements and instrumentalities disclosed. In the drawings:

FIG. 1 shows a cross section schematic view of a first needle array embodiment that is properly attached to skin;

FIG. 2 shows the cross section schematic view of the first needle array embodiment when improperly attached to skin;

FIG. 3 shows a cross section schematic view of a second needle array embodiment that is properly attached to skin;

FIG. 4 shows the cross section schematic view of the second needle array embodiment when improperly attached to skin;

FIG. 5 shows a schematic view of a third needle array embodiment;

FIG. 6 shows a schematic view of a fourth needle array embodiment;

FIG. 7 shows a schematic view of a fifth needle array embodiment;

FIG. 8 shows a schematic view of a sixth needle array embodiment;

FIG. 9 shows a schematic view of a seventh needle array embodiment;

FIG. 10 shows a schematic view of an eighth needle array embodiment;

FIG. 11 shows a schematic view of a ninth needle array embodiment;

FIG. 12 shows a schematic view of a tenth needle array embodiment;

FIG. 13 shows a schematic view of an eleventh needle array embodiment;

FIG. 14 shows a schematic view of a twelfth needle array embodiment;

FIG. 15 shows a schematic view of a thirteenth needle array embodiment;

FIG. 16 shows a schematic view of a fourteenth needle array embodiment;

FIG. 17 shows a schematic view of a fifteenth needle array embodiment;

FIG. 18 shows a schematic view of a sixteenth needle array embodiment;

FIG. 19 shows a schematic view of a seventeenth needle array embodiment;

FIG. 20 shows a schematic view of an eighteenth needle array embodiment;

FIG. 21 shows a schematic view of a nineteenth needle array embodiment;

FIG. 22 shows a schematic view of a twentieth needle array embodiment;

FIG. 23 shows a schematic view of a twenty-first needle array embodiment;

FIG. 24 shows a schematic view of a twenty-second needle array embodiment;

FIG. 25A shows an exploded view of a wearable medicament delivery device;

FIG. 25B shows a pump in an engaged configuration according to an aspect of this disclosure;

FIG. 25C shows the pump of FIG. 25B in a disengaged configuration;

FIG. 26 shows a top view of the wearable medicament delivery device of FIG. 25A before the first housing and second housing are assembled;

FIG. 27 shows a top view of the wearable medicament delivery device of FIG. 25A in an assembled configuration;

FIG. 28 shows a bottom view of the wearable medicament delivery device of FIG. 25A prior to removal of a release sheet from an adhesive patch;

FIG. 29 shows a bottom perspective view of the wearable medicament delivery device of FIG. 25A with the release sheet removed from the adhesive patch;

FIG. 30 shows sensing microneedle data before the sensing microneedles pierce the skin;

FIG. 31 shows sensing microneedle data after the sensing microneedles pierce the skin;

FIG. 32 shows sensing microneedle data collected during a wet injection;

FIG. 33 shows sensing microneedle data collected during an adverse event; and

FIG. 34 shows a process of delivering a medicament using the wearable medicament delivery device.

### DETAILED DESCRIPTION

Some aspects of this invention are directed to needle arrays that can include a plurality of needles thereon. The needle arrays can include one or more injection needles configured to inject, or otherwise deliver, a medicament into the skin at the desired site. The needle array can include injection microneedles for less invasive and less painful medicament administration (as compared to conventional cannula needles) via continuous medicament delivery. The injection microneedles can be hollow and define a lumen therethrough, such that the medicament can be delivered through the lumen. Since microneedles typically do not penetrate deep enough into the skin to touch nerve endings or blood capillaries, the needle arrays according to some aspects of this invention can avoid needle-triggered pain and/or bleeding associated with typical needle cannulas. The term “microneedle” as used herein can mean a micron-scale needle and can include needles with one or more dimensions (e.g., diameter, width, length, etc.) of less than about 1 mm (1000 μm). In embodiments, the term “microneedle” can mean a needle with a length less than about 1 mm (1000 μm) that is targeting an intradermal medicament delivery. The term “cannula needle” as used herein can mean a needle that is larger than a microneedle, such as a needle with a length of 1 mm or greater. Typically, a cannula needle is used for either subcutaneous or intramuscular medicament delivery. Microneedles may be used for more superficial delivery compared to delivery achieved by typical cannula needles.

According to some aspects of this invention, the needle arrays can include sensing needles made of, or coated with, electrically conductive materials. The sensing needles can be sensing microneedles. These sensing microneedles can be used as electrical sensing electrodes for real-time monitoring of the position of the needle array/wearable medicament delivery device on the skin. The sensing microneedles can penetrate the highly resistive stratum corneum skin layer and can contact conductive interstitial fluid in the underlying epidermal areas to provide a more sensitive and accurate signal than, for example, flat electrodes positioned on the surface of the skin only.

In embodiments, tips of sensing microneedles can be spaced (e.g., laterally) from the injection needles to reduce interference from local physiological or medicament-related events. By decoupling and offsetting the sensing microneedles from the injection needle: the sensing microneedles can be formed using less expensive materials and less expensive fabrication techniques; the sensing needles can be formed with different geometries than the injection needles, and such geometries can be optimized for sensing (e.g., such as those described throughout this application and depicted in the drawings); the microneedles can collect data associated with partial detachment of the needle array of medicament delivery device or tilting of the needle array/wearable medicament delivery device away from the skin; signal interference associated with the injection site can be minimized; the number of sensing electrodes can be increased to improve sensitivity using any number of techniques; among other advantages.

The sensitive and accurate data associated with the position of the needle array/wearable medicament delivery device on the skin provided by the sensing microneedles can be used by a controller of the wearable medicament delivery device to determine whether the wearable medicament delivery device is in a proper position on and in the skin for injection. It will be appreciated that the identifiable position can include lateral placement in a 3-dimensional orientation relative to the skin, rotational orientation relative to the skin, and penetrative depth into the skin. If the controller determines that the wearable medicament delivery device is in a proper position for injection based on the data received by the sensing microneedles, the controller can switch to, or remain in, a ready-to-inject state in which the controller will control the wearable medicament delivery device to inject the medicament when instructions to do so are received. If the controller determines that the wearable medicament delivery device is not in a proper position for injection based on the data received by the sensing microneedles, the controller can switch to, or remain in, a default state in which the controller prevents the injection procedure from starting or continuing. The controller can allow the injection to resume after the device has been properly positioned and after the controller receives the data associated with the properly positioned device. This can reduce instances of wet injections (i.e., incomplete injections that cause the medicament to leak onto an external skin surface) that can occur when the wearable medicament delivery device is improperly placed on the skin. Non-limiting examples of improper positioning can include penetration of the sensing microneedles to an insufficient depth into the skin and incorrect rotational orientation (i.e., tilt) of the sensing microneedles. This can also reduce instances of false positives, where the wearable medicament delivery device operates as if a successful injection has occurred; however, due to improper positioning of the device, some or all of the medicament may have actually been dispensed topically on the skin surface rather than either in or under the skin.

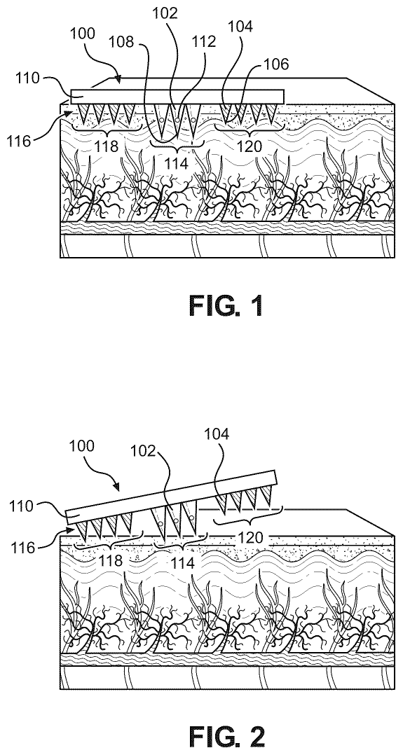

According to some aspects of this invention, the needle arrays can be incorporated into a wearable medicament delivery device that includes a pump that can control delivery of relatively large volumes of medicaments (over 0.5 mL). Medicaments that could be delivered using the wearable medicament delivery device according to some aspects of this invention include, for example, insulin, cancer or oncology treatments, biologic or biosimilar medicaments used to treat chronic conditions, among other possibilities. In embodiments, the wearable medicament delivery device can include a pressure sensor that allows for feedback control of the pump for automatic and fine medicament delivery control of the wearable medicament delivery device. Use of microneedles for injection combined with the pump on the wearable medicament delivery device can allow for high control of closed-loop medicament regimens that could be given via a single instance of delivery or at multiple time points within a day or over multiple days, which can prevent the buildup of drug boluses among other issues. If, or when, the wearable medicament delivery device is detached or dislodge from the skin, or if a user is too active (e.g., exercising vigorously) or too inactive (e.g., asleep) or a in a poor position (e.g., lying prone), the combination of the sensing microneedles with inertial sensors can pause medicament delivery and alert a user (e.g., via a smart phone application) to reconnect, settle, or change activity or position before the dose proceeds. Wearable medicament delivery devices according to some aspects of the invention can provide feedback during self-administration and reduce the frequency or severity of adverse events. According to some aspects of the invention, the wearable medicament delivery device can allow for more targeted and reliable drug delivery to specific routes of administration than conventional wearable medicament delivery devices. These and other aspect of the invention are described further in reference to FIGS. 1-34.

FIG. 1 shows a cross section schematic view of a needle array 100 when the needle array 100 is properly attached to/positioned on the skin of the patient, according to some aspects of the invention. FIG. 2 shows a cross section view of the needle array 100 when the needle array 100 is improperly attached to/positioned on the skin of the patient. The needle array 100 can include an injection needle 102 that can inject a medicament into the patient subcutaneously through the skin. The needle array 100 can include a sensing microneedle 104 that can sense a characteristic associated with the needle array 100. For example, the sensing microneedle 104 can sense a characteristic associated with contact of the sensing microneedle 104 with the skin of the patient. The contact can be with the surface of the skin or with layers of the skin beneath the surface. In some cases, where the sensing microneedle 104 penetrates the skin, the contact between the sensing microneedle 104 and skin can be used to identify or quantity a depth to which the sensing microneedle 104 is placed into the skin of the patient relative to the skin surface. Depth of insertion can be calculated, for example, by measuring the contact between the sensing microneedle 104 and the skin along the length of the sensing microneedle 104. Depth of penetration into the skin can depend on the length of the sensing microneedle 104 and on the position of the sensing microneedle 104 relative to the skin. A tip 106 of the sensing microneedle 104 can be offset (e.g., laterally offset) from a tip 108 of the injection needle 102. According to this configuration, accuracy of the sensing microneedle 104 can be improved since the sensing microneedle 104 can be offset from the injection site and from at least some sources of interference (e.g., liquid from the medicament, localized swelling at the injection site, etc.) associated therewith.

By sensing the characteristic associated with the needle array 100, the sensing microneedle 104 can provide information regarding the state of attachment of the needle array 100 on the skin. For example, and as shown in FIG. 1, when the needle array 100 is properly positioned on the skin (i.e., in a target position on the skin in which the needle array 100 is properly attached to the skin and the microneedle 104 is at the desired depth in the skin for an injection), the sensing microneedle 104 can, in embodiments, penetrate entirely through the stratum corneum layer of skin and sense a characteristic associated with the epidermis or dermis skin layers below the stratum corneum layer. By penetrating entirely through the stratum corneum layer, the sensing microneedle 104 can provide a clear and accurate signal associated with the skin below the stratum corneum layer. This is because the stratum corneum is a dry barrier layer of the skin that has high electrical impedance that can block passage of both medicaments and electrical signals into and out of the body. For sensing purposes, the sensing microneedles 104 can penetrate and overcome the stratum corneum layer and sit within more electrically conductive interstitial fluid in epidermis or dermis layers of the skin. This can generate a larger differential between contact and noncontact states of the sensing microneedles 104 than would be observed using flat electrodes at the surface of the skin that do not penetrate the stratum corneum skin layer. In such aspects, an improper position of the needle array 100 can include, but is not limited to, a position where the sensing microneedles 104 do not sufficiently penetrate the stratum corneum, for example due to incomplete insertion of the sensing microneedles 104 into the skin and/or due to the sensing microneedles 104 being tilted relative to the skin.

In embodiments, the injection needle 102 and the sensing microneedle 104 can each be immovably fixed to a base 110 of the needle array 100. The tip 108 of the injection needle 102 can extend a distance from the base 110 that is at least as far as a distance that the tip 106 of the sensing microneedle 104 extends from the base 110. According to this configuration, when the sensing microneedle 104 senses the signal associated with the skin below the stratum corneum layer, it can be inferred that the injection needle 104 has also penetrated below the stratum corneum layer and therefore that the needle array 100 is in the expected (i.e., proper) position for injection. In some aspects, if the sensing microneedle 104 senses the presence of interstitial fluid, for example, and the tip of the sensing microneedle 104 extends a distance from the base 110 that is the same as or less than a distance that the tip of the injection needle 102 extends, then it can be inferred that the needle array 100 is in the proper (i.e., expected or desired) position relative to the skin.

The injection needle 102 can be hollow with an opening 112 at or near the tip 108 of the injection needle 102. According to this configuration, the medicament can be delivered intradermally into the patient through the injection needle 102 via the opening 112. In embodiments, the opening 112 can be formed in a side of the injection needle 102 offset from the tip 108 of the injection needle 102 towards the base 110 of the needle array 100. This configuration can prevent damage of the injection needle 102 during insertion into the patient or retraction from the patient. In embodiments, the injection needle 102 can be a microneedle. The microneedle can be formed of silicon, stainless steel, glass, ceramic, or polymers such as polycarbonate, polyvinyl pyrrolidone (PVP), and hyaluronic acid (HA), although other materials are possible. An injection needle 102 that is a microneedle can be advantageous for medicament administration because the microneedle can target the intradermal layer of the skin and thereby limit or avoid contact with nerve endings and/or blood capillaries, which can reduce pain and/or bleeding associated with the injection.

In embodiments, the needle array 100 can include only one injection needle 102. In alternative embodiments, the injection needle 102 can be one of a plurality of injection needles 102 arranged in an injection needle array 114 of the needle array 100. The injection needle array 114 can include any number of injection needles 102 including two injection needles 102, three injection needles 102, four injection needles 102, five injection needles 102, or more. Any of the structures, features, or relationships associated with the injection needle 102 described herein can be associated with any of the other injection needles 102 described herein. Accordingly, reference to injection needle 102 herein can apply to any of the injection needles 102 of the injection needle array 114.

The sensing microneedle 104 can be solid, that is, not hollow. The sensing microneedle 104 can be made of an electrically conductive material that can be used to sense the characteristic associated with the needle array 100. In embodiments, the electrically conductive material can be coated over a material with limited conductivity to reduce the quantity of conductive material used on the sensing microneedle 104, which can reduce costs. The electrically conducive material can be an electrically conductive metal such as for example silver, copper, gold, aluminum, zinc, nickel, brass, bronze. Other conductive materials are possible.

In embodiments, the tip 106 of the sensing microneedle 104 can extend a length, measured from the base 110, that is less than a length that the tip 108 of the injection needle 102 extends from the base 110. In some aspects, the length that the tip 106 extends from the base 110 can be less than 1000 μm, less than 700 μm, less than 500 μm, or another suitable length. In embodiments, when the needle array 100 is properly positioned on the skin, as shown in FIG. 1, the sensing microneedle 104 can extend into the skin to a depth of at least 10 μm from the outer surface of the skin.

In embodiments, the needle array 100 can include only one sensing microneedle 104. In alternative embodiments, the sensing microneedle 104 can be one of a plurality of sensing microneedles 104 arranged in a sensing microneedle array 116 of the needle array 100. The sensing microneedle array 116 can include any number of sensing microneedles 104 including two sensing microneedles 104, three sensing microneedles 104, four sensing microneedles 104, five sensing microneedles 104, or more. In embodiments, the sensing microneedle array 116 can include a number of sensing microneedle subarrays. For example, the sensing microneedle array 116 can include a first sensing microneedle subarray 118 and a second sensing microneedle subarray 120. The injection needle 102 and/or the injection needle array 114 can be between the first sensing microneedle subarray 118 and the second sensing microneedle subarray 120. This can allow for symmetrical, continuous sensing of the characteristic associated with the needle array 100 around the injection needle 102 and/or the injection needle array 114 and thereby improve confidence of the inferential position of the injection needle 102 and/or the injection needle array 114 on the skin of the patient. Any of the structures, features, or relationships associated with the sensing microneedles 104 described herein can be associated with any of the other sensing microneedles 104 described herein. Accordingly, reference to the sensing microneedle 104 herein can apply to any of the sensing microneedles 104 of the sensing microneedle array 116, the first sensing microneedle subarray 118, the second sensing microneedle subarray 120, etc.

In embodiments, impedance can be the characteristic associated with the needle array 100 sensed by the sensing microneedle 104. When the needle array 100 is detached from the skin and exposed for example to air surrounding the needle array 100, the sensing microneedle 104 can sense a first impedance value. When the needle array 100 is properly attached to the skin and the sensing microneedle 104 penetrates through the stratum corneum layer of skin, as shown in FIG. 1, the sensing microneedle 104 can sense a second impedance value that is lower than the first impedance value. The second impedance value sensed by the sensing microneedle 104 can be relatively steady when the device is properly positioned. Increases or decreases in the second impedance value can be indicative of an issue with attachment (e.g., as shown in FIG. 2) or with the injection, among other possibilities. It will be appreciated that the acceptable impedance ranges will depend on the materials selected, geometry of the individual needles and/or the needle array, the density of the needles within the needle array, and/or other parameters. Generally, the impedance value measured when the needle array is properly attached to the skin will be lower than the impedance value when the needle array 100 is separated from the skin. The impedance value when the needle array is attached can be multiple magnitudes lower than the impedance value measured when the needle array is detached, for example.

In embodiments, resistance can be a characteristic associated with the needle array 100 sensed by the sensing microneedle 104. When the needle array 100 is detached from the skin and exposed for example to air surrounding the needle array 100, the sensing microneedle 104 can sense a first resistance value. When the needle array 100 is properly attached to the skin and the sensing microneedle 104 penetrates through the stratum corneum layer of skin, as shown in FIG. 1, the sensing microneedle 104 can sense a second resistance value that is lower than the first resistance value. The second resistance value sensed by the sensing microneedle 104 can be relatively steady when the device is properly positioned. Increases or decreases in the second resistance value can be indicative of an issue with attachment (e.g., as shown in FIG. 2) or with injection, among other possibilities. It will be appreciated that the acceptable resistance ranges will depend on the materials selected, geometry of the individual needles and/or the needle array, the density of the needles within the needle array, and/or other parameters. In some non-limiting aspects, when the needle array 100 is attached to the skin, the measured resistance can range from approximately 1-10 kOhm; when the needle array 100 is detached from the skin, the resistance would be magnitudes greater.

In embodiments, capacitance can be the characteristic associated with the needle array 100 sensed by the sensing microneedle 104. When the needle array 100 is detached from the skin and exposed for example to air surrounding the needle array 100, the sensing microneedle 104 can sense a first capacitance value. When the needle array 100 is properly attached to the skin and the sensing microneedle 104 penetrates through the stratum corneum layer of skin, as shown in FIG. 1, the sensing microneedle 104 can sense a second capacitance value that is higher than the first capacitance value. The second capacitance value sensed by the sensing microneedle 104 can be relatively steady when the device is properly positioned. Decreases or increases in the second capacitance value can be indicative of an issue with attachment (e.g., as shown in FIG. 2) or with injection, among other possibilities. It will be appreciated that the acceptable capacitance ranges will depend on the materials selected, geometry of the individual needles and/or the needle array, the density of the needles within the needle array, and/or other parameters.

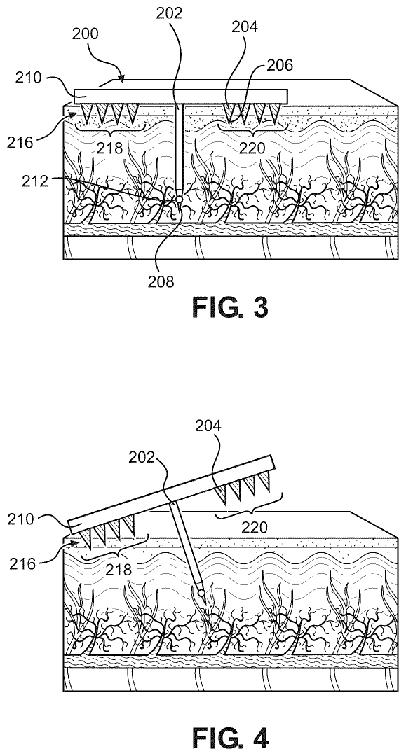

FIG. 3 shows a cross section schematic view of a needle array 200 when the needle array 200 is properly attached to/positioned on the skin of the patient, according to some aspects of the invention. FIG. 4 shows a cross section view of the needle array 200 when the needle array 200 is improperly attached to/positioned on the skin of the patient. Except where clearly contradictory, the needle array 200 can include any or all of the features, structures, and relationships described with respect to the needle array 100 including, for example, the injection needle 202 with the tip 208 and the opening 212, the sensing microneedle 204 with the tip 206, the base 210, and the sensing microneedle array 216 with the first sensing microneedle subarray 218 and the second sensing microneedle subarray 220. The injection needle 202 of the needle array 200 can be a cannula needle. An injection needle 202 that is a cannula needle can be advantageous for medicament administration because it can penetrate further into the skin than a microneedle, which is necessary for delivering medicaments either subcutaneously or intramuscularly.

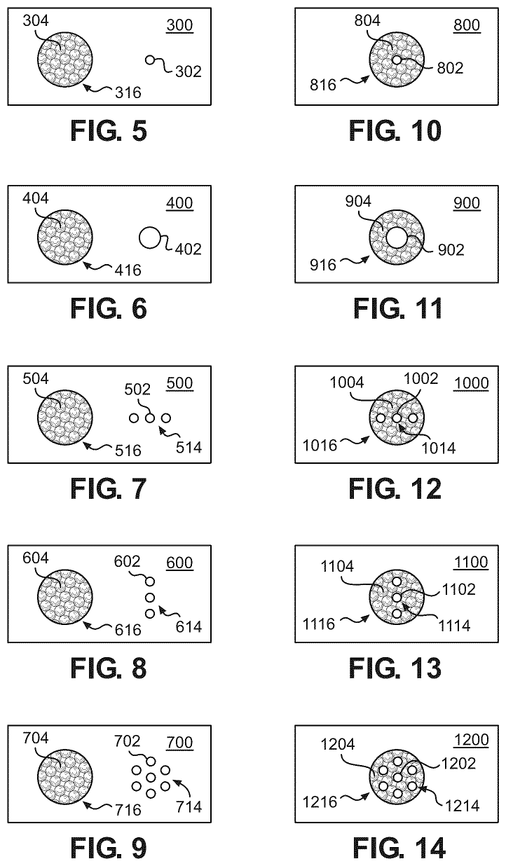

According to some aspects of this invention, a number of different needle arrays with different injection needle(s) and sensing microneedles are possible. FIGS. 5-24 show some such needle arrays, though others are possible. Except where clearly contradictory, the needle arrays 300, 400, 500, 600, 700, 800, 900, 1000, 1100, 1200, 1300, 1400, 1500, 1600, 1700, 1800, 1900, 2000, 2100, 2200 shown in FIGS. 5-24 can include any of the features, structures, and relationships of the needle arrays 100, 200, and vice versa.

FIG. 5 shows a schematic view of a needle array 300 according to some aspects of the invention. The needle array 300 can include one injection needle 302 that can be a microneedle. The needle array 300 can include a single sensing microneedle array 316 with a plurality of sensing microneedles 304 that are each offset from the injection needle 302.

FIG. 6 shows a schematic view of a needle array 400 according to some aspects of the invention. The needle array 400 can include one injection needle 402 that can be a cannula needle. The needle array 400 can include a single sensing microneedle array 416 with a plurality of sensing microneedles 404 that are each offset from the injection needle 402.

FIG. 7 shows a schematic view of a needle array 500 according to some aspects of the invention. The needle array 500 can include one injection needle array 514 with three injection needles 502 arranged in a 1 by 3 horizontal array. The injection needles 502 can be microneedles. The needle array 500 can include a single sensing microneedle array 516 with a plurality of sensing microneedles 504 that are each offset from each of the injection needles 502 of the injection needle array 514.

FIG. 8 shows a schematic view of a needle array 600 according to some aspects of the invention. The needle array 600 can include one injection needle array 614 with three injection needles 602 arranged in a 3 by 1 vertical array. The injection needles 602 can be microneedles. The needle array 600 can include a single sensing microneedle array 616 with a plurality of sensing microneedles 604 that are each offset from each of the injection needles 602 of the injection needle array 614.

FIG. 9 shows a schematic view of a needle array 700 according to some aspects of the invention. The needle array 700 can include one injection needle array 714 with seven injection needles 702 arranged in a cluster. The injection needles 702 can be microneedles. The needle array 700 can include a single sensing microneedle array 716 with a plurality of sensing microneedles 704 that are each offset from each of the injection needles 702 of the injection needle array 714.

FIG. 10 shows a schematic view of a needle array 800 according to some aspects of the invention. The needle array 800 can include one injection needle 802 that can be a microneedle. The needle array 800 can include one sensing microneedle array 816 with a plurality of sensing microneedles 804 that are each offset from the injection needle 802. The injection needle 802 can be concentric with the sensing microneedle array 816.

FIG. 11 shows a schematic view of a needle array 900 according to some aspects of the invention. The needle array 900 can include one injection needle 902 that can be a cannula needle. The needle array 900 can include one sensing microneedle array 916 with a plurality of sensing microneedles 904 that are each offset from the injection needle 902. The injection needle 902 can be concentric with the sensing microneedle array 916.

FIG. 12 shows a schematic view of a needle array 1000 according to some aspects of the invention. The needle array 1000 can include one injection needle array 1014 with three injection needles 1002 arranged in a 1 by 3 horizontal array. The injection needles 1002 can be microneedles. The needle array 1000 can include one sensing microneedle array 1016 with a plurality of sensing microneedles 1004 that are each offset from each of the injection needles 1002 of the injection needle array 1014. The injection needle array 1014 can be concentric with the sensing microneedle array 1016.

FIG. 13 shows a schematic view of a needle array 1100 according to some aspects of the invention. The needle array 1100 can include one injection needle array 1114 with three injection needles 1102 arranged in a 3 by 1 vertical array. The injection needles 1102 can be microneedles. The needle array 1100 can include one sensing microneedle array 1116 with a plurality of sensing microneedles 1104 that are each offset from each of the injection needles 1102 of the injection needle array 1114. The injection needle array 1114 can be concentric with the sensing microneedle array 1116.

FIG. 14 shows a schematic view of a needle array 1200 according to some aspects of the invention. The needle array 1200 can include one injection needle array 1214 with seven injection needles 1202 arranged in a cluster. The injection needles 1202 can be microneedles. The needle array 1200 can include one sensing microneedle array 1216 with a plurality of sensing microneedles 1204 that are each offset from each of the injection needles 1202 of the injection needle array 1214. The injection needle array 1214 can be concentric with the sensing microneedle array 1216.

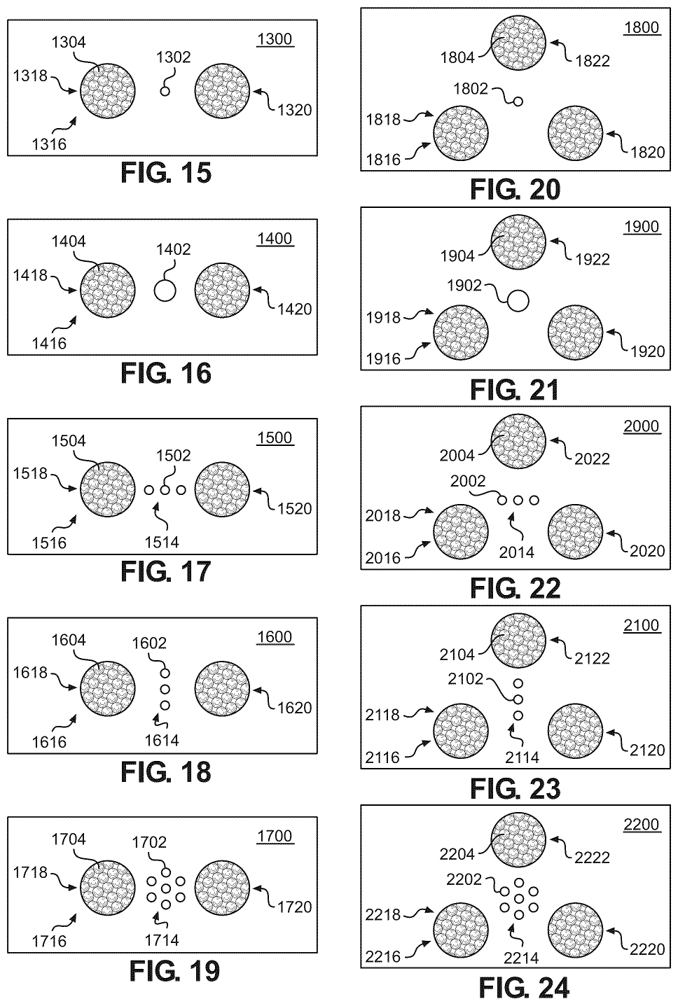

FIG. 15 shows a schematic view of a needle array 1300 according to some aspects of the invention. The needle array 1300 can include one injection needle 1302 that can be a microneedle. The needle array 1300 can include a sensing microneedle array 1316 with a plurality of sensing microneedles 1304 that are each offset from the injection needle 1302. The sensing microneedle array 1316 can include a first sensing microneedle subarray 1318 and a second sensing microneedle subarray 1320. The injection needle 1302 can be between the first sensing microneedle subarray 1318 and the second sensing microneedle subarray 1320. In embodiments, injection needle 1302 can be spaced equidistantly between the first sensing microneedle subarray 1318 and the second sensing microneedle subarray 1320.

FIG. 16 shows a schematic view of a needle array 1400 according to some aspects of the invention. The needle array 1400 can include one injection needle 1402 that can be a cannula needle. The needle array 1400 can include a sensing microneedle array 1416 with a plurality of sensing microneedles 1404 that are each offset from the injection needle 1402. The sensing microneedle array 1416 can include a first sensing microneedle subarray 1418 and a second sensing microneedle subarray 1420. The injection needle 1402 can be between the first sensing microneedle subarray 1418 and the second sensing microneedle subarray 1420. In embodiments, the injection needle 1402 can be spaced equidistantly between the first sensing microneedle subarray 1418 and the second sensing microneedle subarray 1420.

FIG. 17 shows a schematic view of a needle array 1500 according to some aspects of the invention. The needle array 1500 can include one injection needle array 1514 with three injection needles 1502 arranged in a 1 by 3 horizontal array. The injection needles 1502 can be microneedles. The needle array 1500 can include a sensing microneedle array 1516 with a plurality of sensing microneedles 1504 that are each offset from each of the injection needles 1502. The sensing microneedle array 1516 can include a first sensing microneedle subarray 1518 and a second sensing microneedle subarray 1520. The injection needle array 1514 can be between the first sensing microneedle subarray 1518 and the second sensing microneedle subarray 1520. In embodiments, the injection needle array 1514 can be spaced equidistantly between the first sensing microneedle subarray 1518 and the second sensing microneedle subarray 1520.

FIG. 18 shows a schematic view of a needle array 1600 according to some aspects of the invention. The needle array 1600 can include one injection needle array 1614 with three injection needles 1602 arranged in a 3 by 1 vertical array. The injection needles 1602 can be microneedles. The needle array 1600 can include a sensing microneedle array 1616 with a plurality of sensing microneedles 1604 that are each offset from the injection needles 1602. The sensing microneedle array 1616 can include a first sensing microneedle subarray 1618 and a second sensing microneedle subarray 1620. The injection needle array 1614 can be between the first sensing microneedle subarray 1618 and the second sensing microneedle subarray 1620. In embodiments, the injection needle array 1614 can be spaced equidistantly between the first sensing microneedle subarray 1618 and the second sensing microneedle subarray 1620.

FIG. 19 shows a schematic view of a needle array 1700 according to some aspects of the invention. The needle array 1700 can include one injection needle array 1714 with seven injection needles 1702 arranged in a cluster. The injection needles 1702 can be microneedles. The needle array 1700 can include a sensing microneedle array 1716 with a plurality of sensing microneedles 1704 that are each offset from each of the injection needles 1702. The sensing microneedle array 1716 can include a first sensing microneedle subarray 1718 and a second sensing microneedle subarray 1720. The injection needle array 1714 can be between the first sensing microneedle subarray 1718 and the second sensing microneedle subarray 1720. In embodiments, the injection needle array 1714 can be spaced equidistantly between the first sensing microneedle subarray 1718 and the second sensing microneedle subarray 1720.

FIG. 20 shows a schematic view of a needle array 1800 according to some aspects of the invention. The needle array 1800 can include one injection needle 1802 that can be a microneedle. The needle array 1800 can include a sensing microneedle array 1816 with a plurality of sensing microneedles 1804 that are each offset from the injection needle 1802. The sensing microneedle array 1816 can include a first sensing microneedle subarray 1818, a second sensing microneedle subarray 1820, and a third sensing microneedle subarray 1822. The injection needle 1802 can be between the first sensing microneedle subarray 1818, the second sensing microneedle subarray 1820, and the third sensing microneedle subarray 1822. In embodiments, the injection needle 1802 can be spaced equidistantly between the first sensing microneedle subarray 1818, the second sensing microneedle subarray 1820, and the third sensing microneedle subarray 1822.

FIG. 21 shows a schematic view of a needle array 1900 according to some aspects of the invention. The needle array 1900 can include one injection needle 1902 that can be a cannula needle. The needle array 1900 can include a sensing microneedle array 1916 with a plurality of sensing microneedles 1904 that are each offset from the injection needle 1902. The sensing microneedle array 1916 can include a first sensing microneedle subarray 1918, a second sensing microneedle subarray 1920, and a third sensing microneedle subarray 1922. The injection needle 1902 can be between the first sensing microneedle subarray 1918, the second sensing microneedle subarray 1920, and the third sensing microneedle subarray 1922. In embodiments, the injection needle 1902 can be spaced equidistantly between the first sensing microneedle subarray 1918, the second sensing microneedle subarray 1920, and the third sensing microneedle subarray 1922.

FIG. 22 shows a schematic view of a needle array 2000 according to some aspects of the invention. The needle array 2000 can include one injection needle array 2014 with three injection needles 2002 arranged in a 1 by 3 horizontal array. The injection needles 2002 can be microneedles. The needle array 2000 can include a sensing microneedle array 2016 with a plurality of sensing microneedles 2004 that are each offset from each of the injection needles 2002. The sensing microneedle array 2016 can include a first sensing microneedle subarray 2018, a second sensing microneedle subarray 2020, and a third sensing microneedle subarray 2022. The injection needle array 2014 can be between the first sensing microneedle subarray 2018, the second sensing microneedle subarray 2020, and the third sensing microneedle subarray 2022. In embodiments, the injection needle array 2014 can be spaced equidistantly between the first sensing microneedle subarray 2018, the second sensing microneedle subarray 2020, and the third sensing microneedle subarray 2022.

FIG. 23 shows a schematic view of a needle array 2100 according to some aspects of the invention. The needle array 2100 can include one injection needle array 2114 with three injection needles 2102 arranged in a 3 by 1 vertical array. The injection needles 2102 can be microneedles. The needle array 2100 can include a sensing microneedle array 2116 with a plurality of sensing microneedles 2104 that are each offset from each of the injection needles 2102. The sensing microneedle array 2116 can include a first sensing microneedle subarray 2118, a second sensing microneedle subarray 2120, and a third sensing microneedle subarray 2122. The injection needle array 2114 can be between the first sensing microneedle subarray 2118, the second sensing microneedle subarray 2120, and the third sensing microneedle subarray 2122. In embodiments, the injection needle array 2114 can be spaced equidistantly between the first sensing microneedle subarray 2118, the second sensing microneedle subarray 2120, and the third sensing microneedle subarray 2122.

FIG. 24 shows a schematic view of a needle array 2200 according to some aspects of the invention. The needle array 2200 can include one injection needle array 2214 with seven injection needles 2202 arranged in a cluster. The injection needles 2202 can be microneedles. The needle array 2200 can include a sensing microneedle array 2216 with a plurality of sensing microneedles 2204 that are each offset from the injection needles 2202. The sensing microneedle array 2216 can include a first sensing microneedle subarray 2218, a second sensing microneedle subarray 2220, and a third sensing microneedle subarray 2222. The injection needle array 2214 can be between the first sensing microneedle subarray 2218, the second sensing microneedle subarray 2220, and the third sensing microneedle subarray 2222. In embodiments, the injection needle array 2214 can be spaced equidistantly between the first sensing microneedle subarray 2218, the second sensing microneedle subarray 2220, and the third sensing microneedle subarray 2222.

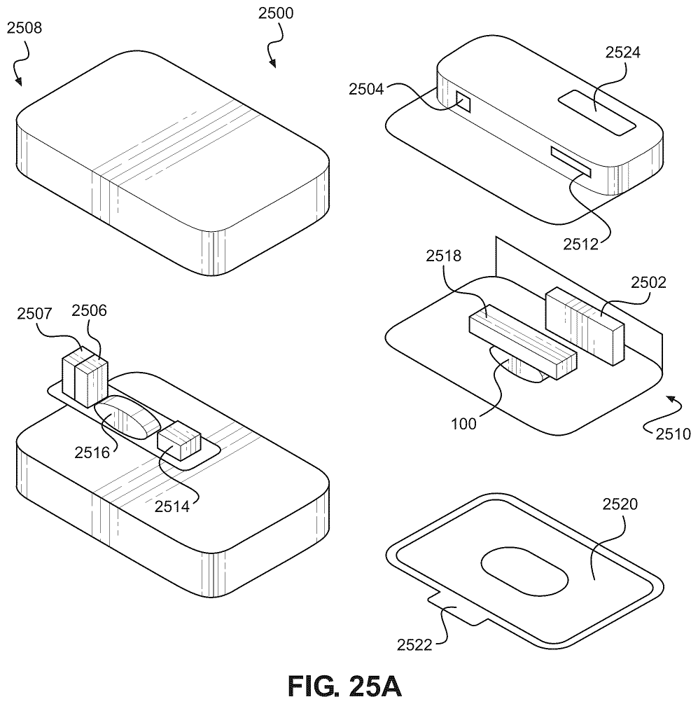

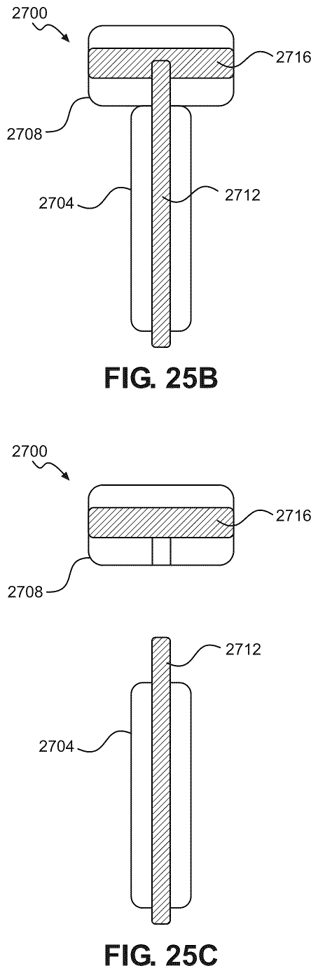

FIG. 25A shows an exploded view of a wearable medicament delivery device 2500 according to some aspects of the invention. The wearable medicament delivery device 2500 can include the needle array 100, as previously described. In embodiments, the wearable medicament delivery device 2500 can include any of the needle arrays 200, 300, 400, 500, 600, 700, 800, 900, 1000, 1100, 1200, 1300, 1400, 1500, 1600, 1700, 1800, 1900, 2000, 2100, or 2200 previously described. The wearable medicament delivery device 2500 can include a container 2502 that can contain the medicament. In embodiments, the container 2502 can be flexible, for example a bladder, bag, pouch, etc. In embodiments, the container 2502 can be transparent and/or translucent, which can allow for visual inspection of the medicament contained therein. In embodiments, the container 2502 can contain over 0.5 mL of the medicament or of a medicament solution. The wearable medicament delivery device 2500 can include a pump 2504 that can be operatively connected to the injection needle 102 and to the container 2502. The pump 2504 can pump the medicament from the container 2502 and through the injection needle 102 to inject the medicament and to the patient. The pump 2504 can be any suitable pump, for example a peristaltic pump. The wearable medicament delivery device 2500 can include a motor 2507 configured to operate the pump 2504. The motor 2507 can cause rotation of the pump 2504 in one or two directions. The wearable medicament delivery device 2500 can include a controller 2506 that can be operatively connected to the pump 2504 and to the sensing microneedle 104. The motor 2507 can be operably connected to the controller 2506 and can be actuated by the controller 2506. The controller 2506 can actuate the motor 2507 to operate the pump 2504 to deliver an entirety of the medicament contained in the container 2502. In a default state, the controller 2506 can prevent the pump 2504 from pumping the medicament from the container 2502 to the injection needle 102 when the characteristic associated with the depth of the sensing microneedle 104 is above a threshold value, as described further later. In a ready-to-inject state, the controller 2506 can control the pump 2504 to pump the medicament from the container 2502 to the injection needle 102 when instructed to do so by a user.

In embodiments, the wearable medicament delivery device 2500 can include a first housing 2508 and the second housing 2510. The first housing 2508 can be reusable and can house high value components of the wearable medicament delivery device 2500 that do not directly contact the medicament or other structures that make direct contact with the injection site of the patient. For example, the first housing 2508 can house the controller 2506, among other structures. According to this configuration, costs associated with reuse of the wearable medicament delivery device 2500 can be reduced since at least some components of the wearable medicament delivery device 2500 can be used again. The second housing 2510 can be disposable and can house components of the wearable medicament delivery device 2500 that directly contact the medicament or other structures that directly contact the injection site of the patient. For example, the second housing 2510 can house the pump 2504, the container 2502, and the needle array 100, among other structures. According to this configuration, contamination of the wearable medicament delivery device 2500 can be prevented or reduced since components of the wearable medicament delivery device 2500 that directly contact the medicament or other structures that directly contact the patient can be used only once. The first housing 2508 and the second housing 2510 can be removably connectable to each other via a number of different techniques including snap fitting, magnets, and/or with fasteners.

In embodiments, the second housing 2510 can include an electrical connector 2512 that can interface with a complementary electrical connector of the first housing 2508. In embodiments, when the electrical connector 2512 interfaces with the complementary electrical connector of the first housing 2508, the controller 2506 can actuate fasteners to removably connect the first housing 2508 to the second housing 2510. The electrical connector 2512 can also be operatively connected to, for example, the pump 2504, the sensing microneedles 104, among other structures. Electrical connector 2512 can electrically connect the pump 2504, the sensing microneedles 104, among other structures, to the controller 2506 to allow the controller 2506 to electrically control such structures.

The wearable medicament delivery device 2500 can include a power supply 2514 such as, for example, a battery. The power supply 2514 can be operatively connected to and can supply power to any or all of the electrical structures of the wearable medicament delivery device 2500. In embodiments, the power supply 2514 can be housed within the first housing 2508.

The wearable medicament delivery device 2500 can include a user interface 2516, such as a button, which can allow the user to initiate the injection and self-administer the medicament. The user interface 2516 can be operatively connected to the controller 2506, the power supply 2514, among other structures. In embodiments, the user interface 2516 can be housed within the first housing 2508.

In embodiments, the wearable medicament delivery device 2500 can include a pressure sensor 2518. The pressure sensor 2518 can be fluidly connected in the flow path between the container 2502 and the injection needle 102. The pressure sensor 2518 can be electrically connected to the controller 2506. The controller 2506 can receive pressure data sensed by the pressure sensor 2518 and can use the pressure data to control the pump and regulate injection of the medicament into the patient. In embodiments, the pressure sensor 2518 can be housed within the second housing 2510. In embodiments, the wearable medicament delivery device 2500 can include other sensors (e.g., tilt sensors, inertial sensors, etc.) and the controller 2506 can use data from such other sensors to evaluate whether the device is properly positioned on (and remains on) the patient, that the microneedle is at (and remains at) the desired depth in the skin, that the patient is in (and remains in) the desired position and the patient is at (and remains at) the desired level of activity before and during an injection.

The wearable medicament delivery device 2500 can include an adhesive patch 2520 with a release sheet 2522 that is removable. When the release sheet 2522 is removed, the adhesive of the adhesive patch 2520 can be exposed and the wearable medicament delivery device 2500 can be a fixed to the injection site of the patient. In embodiments, additional or alternative means for fixing the wearable medicament delivery device 2500 to the patient can be provided such as for example straps. In embodiments, the adhesive patch 2520 can be fixed to the second housing 2510.

In embodiments, the second housing 2510 can include a window 2524. The window 2524 can be arranged over the container 2502 to allow for visual inspection of the container 2502 through the wearable medicament delivery device 2500.

The pump 2504 can include any suitable pump that can be configured to move a liquid medicament from a source to a destination. In such cases, where the pump 2504 is entirely on or in the second housing 2510, the pump 2504 may be disposable along with the second housing 2510. In some aspects, the pump utilized in such devices can be designed to similarly have reusable and disposable portions. Reusing at least a portion of the pumping mechanism can reduce waste and cost associated with the delivery device. Referring to FIGS. 25B and 25C, a pump 2700 can contain a first portion 2704 and a second portion 2708 configured to releasably engage with the first portion 2704. While the pump 2700 is described herein with reference to the wearable medicament delivery device 2500, it will be understood that the pump 2700 can be used in any other suitable wearable delivery device having at least two separable portions. The first portion 2704 may be disposed on or in a first housing of a delivery device, for example the first housing 2508 of the wearable medicament delivery device 2500, and the second portion 2704 may be disposed on or in a second housing, for example the second housing 2510 of the wearable medicament delivery device 2500. As such, the first portion 2704 may be designed to be reusable, while the second portion 2708 may be designed to be disposable.

The first portion 2704 may include a rotatable pump shaft 2712. Rotation of the pump shaft 2712 can be actuated by a motor, such as the motor 2507 described above. The second portion 2708 can include a rotatable pump head 2716 configured to releasably engage with and be rotated by the pump shaft 2712. The pump head 2716 can be configured to contact the medicament being pumped directly, or, alternatively, to contact a deformable tube through which the medicament can flow. Neither the pump shaft 2712 nor any other component of the first portion 2704 contacts the medicament or the medicament tube. As such, these components can be reused with low risk of contamination and without requiring sterilization. The pump head 2716 and other components of the second portion 2708 can contact the medicament or the medicament tube, and as such have a higher standard for cleanliness. These components can be designed to be discarded after a single use of the medicament delivery device.

During use, the first portion 2704 can be contacted with the second portion 2708 such that the pump shaft 2712 engages with the pump head 2716, such that when the pump shaft 2712 is rotated, the pump head 2716 also rotates. The pump 2700 in such an engaged configuration is shown in FIG. 25B. In some aspects, the pump shaft 2712 may be keyed to have a specific geometry that corresponds to a complementary geometry defined on the pump head 2716, such that the pump shaft 2712 can only be engaged with the pump head 2716 in a desired orientation relative to the pump head 2716. The first portion 2704 can be configured to releasably engage with, and disengage from, different second portions 2708. After injection is complete, the first and second portions 2704 and 2708 can be separated from each other, and the second portion 2708 can be disposed. The first portion 2704 can be used again with a different second portion 2708. The first and second portions 2704 and 2708 are shown disengaged in FIG. 25C.

In some aspects, for example when used with the wearable medicament delivery device 2500, the first portion 2704 of the pump 2700 can be engaged with the second portion 2708 when the first housing 2508 is engaged with the second housing 2510. After use, the first housing 2508 can be separated from the second housing 2510, thus also separating the first and second portions 2704 and 2708 of the pump 2700.

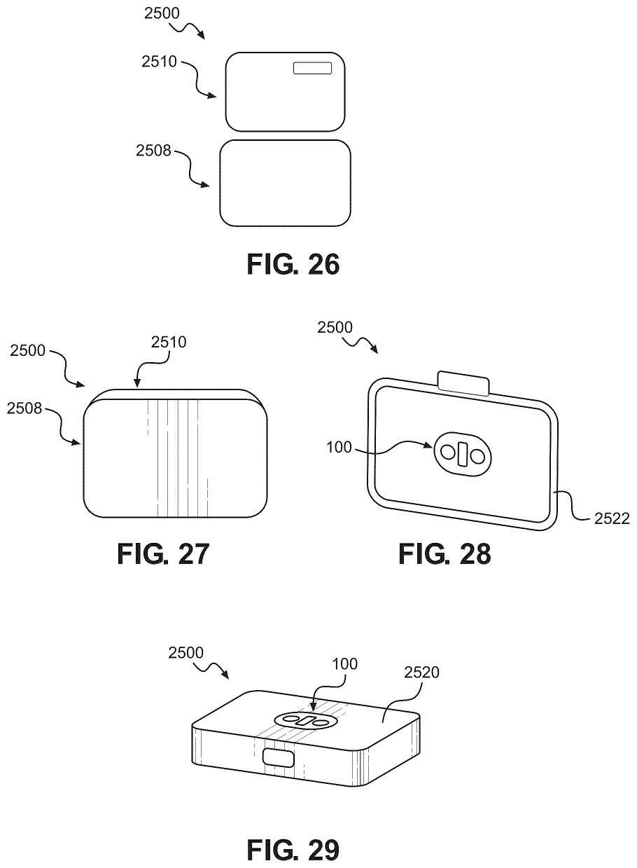

FIGS. 26-29 show stages of preparation of the wearable medicament delivery device 2500 for attachment to the patient according to some aspects of the invention. FIG. 26 shows a top view of the wearable medicament delivery device 2500 before the first housing 2508 and the second housing 2510 are assembled together. FIG. 27 shows a top view of the wearable medicament delivery device 2500 in an assembled configuration in which the first housing 2508 and the second housing 2510 are removably connected to each other. In embodiments, the first housing 2508 can include an indicator that audially or visually indicates that the device is in the assembled configuration. FIG. 28 shows a bottom view of the wearable medicament delivery device 2500 in the assembled configuration with the release sheet 2522 attached. FIG. 29 shows the bottom view of the wearable medicament delivery device 2500 and the assembled configuration with the release sheet 2522 removed and with the wearable medicament delivery device 2500 ready to be attached to the injection site of the patient.

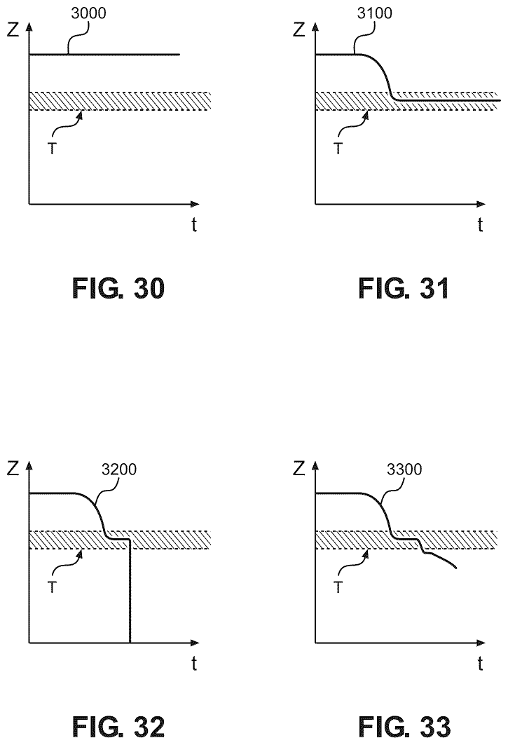

FIGS. 30-33 show example data that can be collected from the sensing microneedles 104 and processed by the controller 2506. The example data shown in FIGS. 30-33 can be impedance measurements over time, for example at 1 kHz, though other data can be collected including for example capacitance and resistance data. FIG. 30 shows first impedance data 3000 collected over time when the wearable medicament delivery device 2500 is not connected to the patient or not properly connected to the patient. The first impedance data 3000 corresponding to an open circuit is consistently above a target range of impedance values T that represents the expected impedance within interstitial fluid of the skin below the stratum corneum layer. The first impedance data 3000 may be that of air, for example greater than about 1 MΩ. The target range of impedance T can be between about 1-10 kΩ. When the controller 2506 receive data signals from the sensing microneedles 104 similar to the first impedance data 3000, i.e., consistently above the target range T, the controller 2506 can determine that the wearable medicament delivery device is not connected to the patient or is not properly attached to the patient and can prevent the pump 2504 from pumping the medicament. As noted elsewhere, the measured impedance ranges can depend on the materials selected, geometry of the individual needles and/or the needle array, the density of the needles within the needle array, and/or other parameters.

FIG. 31 shows second impedance data 3100 collected over time when the wearable medicament delivery device 2500 is properly connected to the patient. The second impedance data 3100 drops and then levels off within the target range T. This can indicate that the sensing microneedles 104 are likely in contact with the interstitial fluid of the skin below the stratum corneum layer. This can also indicate that, due to previously described structural arrangement of the needle array 100, the injection needle 102 is also in contact with the interstitial fluid of the skin below the stratum corneum layer and has penetrated the skin at least as deep as the sensing microneedles 104 or deeper. The controller 2506, when receiving data signals similar to the second impedance data 3100, i.e., consistently within the target range T, can automatically switch to the ready-to-inject state and thereby allow the pump 2504 to pump the medicament when instructed to do so by a user.

FIG. 32 shows third impedance data 3200 collected over time during a wet injection. The third impedance data 3200 drops to within the target range T, indicating that the sensing microneedles 104 are at least at some point in contact with the interstitial fluid of the skin below the stratum corneum layer. The third impedance data 3200 then precipitously drops below the target range T, which can indicate a wet injection in which fluid from the medicament shorts out the sensing microneedles 104. The controller 2506, when receiving data signals similar to the third impedance data 3200, can automatically return to the default state thereby pausing the pump 2504. In embodiments, the controller 2506 can indicate to a user that an injection fault may have occurred.

FIG. 33 shows fourth impedance data 3300 collected over time during a potential adverse event, such as swelling at the injection site. The fourth impedance data 3300 drops to within the target range T, indicating that the sensing microneedles 104 are at least at some point in contact with the interstitial fluid of the skin below the stratum corneum layer. The fourth impedance data 3300 then drops below the target range T, which can indicate occurrence of a potential adverse event (e.g., swelling at the injection site). The controller 2506, when receiving data signals similar to the fourth impedance data 3300, can automatically return to the default state thereby pausing the pump 2504. In embodiments, the controller 2506 can indicate to a user that an injection fault may have occurred.

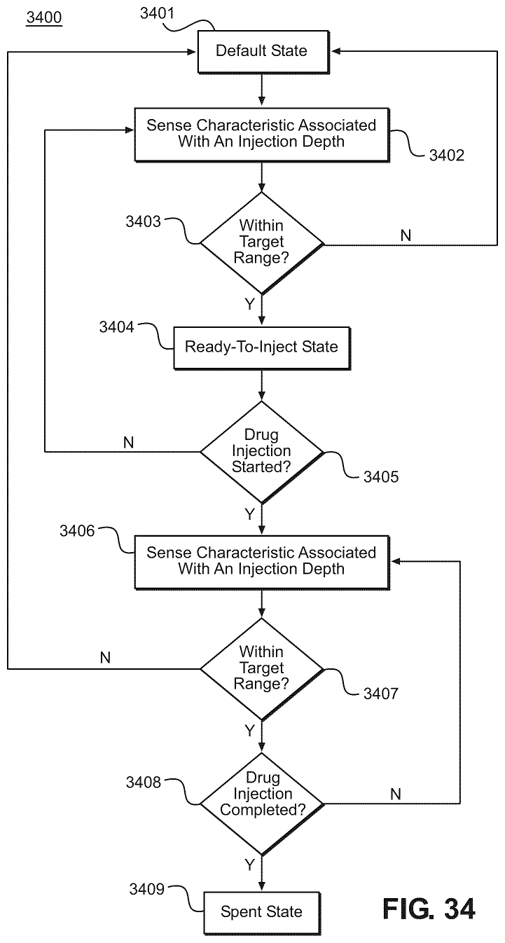

FIG. 34 shows a process 3400 of delivering a medicament according to some aspects of the invention. The process 3400 can deliver the medicament using the wearable medicament delivery device 2500, as previously described. The process 3400 can begin, at step 3401, with the wearable medicament delivery device 2500 in a default state. In the default state, the controller 2506 can prevent operation of the pump 2504 to prevent delivery of the medicament to the injection needle 102. For example, in the default state, the controller 2506 will not activate the pump 2504 in response to user instructions to activate the pump by pressing the user interface 2516. The default state can thus be a safety state the prevents injection unless the controller 2506 receives an indication for the sensing microneedles 104 that the wearable medicament delivery device 2500 is properly positioned. The controller 2506 can automatically default to the default state, for example, when the wearable medicament delivery device 2500 is activated. The wearable medicament delivery device 2500 can be automatically activated, for example, when the first housing 2508 is removably connected to the second housing 2510, as shown in FIGS. 26 and 27.

The process 3400 can include, at step 3402, sensing the characteristic associated with the wearable medicament delivery device 2500. The characteristic can be sensed with the sensing microneedles 104, as previously described. The characteristic can be associated with the depth that the sensing microneedles 104 penetrate into the skin, as previously described. In some aspects, the characteristic can be associated with any other parameter described throughout this application, such as lateral position and/or the tilt of the sensing microneedles 104 relative to the surface of the skin. The process 3400 can include multiple iterations of step 3402, at which different characteristics may be sensed. It will be understood that the multiple steps 3402 can occur simultaneously or serially in any suitable order. The process 3400 can include sensing 1, 2, 3, . . . 5, or any other desired number of characteristics.

The process 3400 can include, at step 3403, determining using the controller 2506 whether the characteristic sensed at step 3402 is within the target range, as previously described. The target range can be an impedance, resistance, or capacitance range associated with a particular depth in the skin surface at the injection site of the patient, such as for example below the stratum corneum layer. If the controller 2506 determines that the characteristic is within the target range, the process 3400 can proceed to step 3404. If the controller 2506 determines that the characteristic is not within the target range, the process 3400 can loop back to step 3401 and the wearable medicament delivery device 2500 can remain in the default state until the controller 2506 determines that the characteristic is within the target range.

The process 3400 can include, at step 3404, automatically switching the controller 2506 from the default state to a ready-to-inject state, or, maintaining the ready-to-inject state if the controller 2506 is already in the ready-to-inject state. In the ready-to-inject state, the controller 2506 can activate the pump 2504 upon receipt of instructions to do so from the user interface 2516. For example, when a user actuates the user interface 2516 (e.g., presses a button) and the controller 2506 is in the ready-to-inject state, the controller 2506 will automatically activate the pump 2504 to pump the medicament through the injection needle 102. In embodiments, the controller 2506 can control the pump 2504 based on feedback from the pressure sensor 2518. Because the sensing microneedles 104 sensed that the characteristic is within the target range at step 3403, it can be inferred that the injection needle 102 is also properly positioned for the injection, as previously described.

The process 3400 can include, at step 3405, determining using the controller 2506 whether the injection has started. The controller 2506 can determine whether the injection started based upon whether the controller 2506 has received the signal to start the injection from the user interface 2516 and by monitoring the pump 2504. If the controller 2506 determines that the injection has started, the process 3400 can proceed to step 3406. If the controller 2506 determines that the injection has not started, the process 3400 can loop back to step 3402.

The process 3400 can include, at step 3406, sensing the characteristic associated with the wearable medicament delivery device 2500. Step 3406 can be substantially the same as step 3402 but for its order in the process 3400.

The process 3400 can include, at step 3407, determining with the controller 2506 whether the characteristic sensed at step 3406 is within the target range. Step 3407 can be substantially the same as step 3403 but for its order in the process 3400. If the controller 2506 determines that the characteristic is within the target range, the process 3400 can proceed to step 3408. If not, the process can loop back to step 3402 and effectively pause the injection. The injection can then continue from where it left off once the wearable medicament delivery device 2500 is repositioned and the process 3400 proceeds from step 3402 again.

The process 3400 can include, at step 3408, determining using the controller 2506 whether the medicament injection has completed. The controller 2506 can determine whether the medicament injection has been completed for example based upon feedback from the pressure sensor 2518 and/or known amounts of medicament to be injected, which can be stored in a memory of the controller 2506. If the controller 2506 determines that the injection has completed the controller 2506 can switch to a spent state at step 3409 of the process 3400. In the spent state, the controller 2506 can indicate that the device has been used and prevent further control of the components of the spent second housing 2510. For example, in the spent state the controller 2506 will not operate the pump 2504 regardless of the status of the sensing microneedles 104 and regardless of instructions received from the user interface 2516. In embodiments, the controller 2506 can return to the default state when another second housing 2510 is removably attached to the first housing 2508, effectively restarting the process 3400 from the beginning. If the controller 2506 determines that the injection has not been completed, the process 3400 can loop back to step 3406. Steps 3406, 3407, and 3408 can allow for continuous monitoring of the depth of the sensing microneedles 104 during the entirety of the injection. This can improve confidence that the injection needle 102 remains in a proper position and/or can detect potential adverse events associated with the medicament injection, as shown in FIGS. 32 and 33 and described previously.

The process 3400 may include one or more optional steps during which the user can be alerted to one or more statuses or status changes of the wearable medicament delivery device 2500. The alerts can include visual, auditory, and/or haptic signals. These one or more optional steps may occur at any point during the process 3400 during, before, or after any of the steps described above.

The process 3400 may include one or more optional steps during which the user can be alerted to one or more statuses or status changes of the wearable medicament delivery device 2500. The alerts can include visual, auditory, and/or haptic signals. These one or more optional steps may occur at any point during the process 3400 during, before, or after any of the steps described above

It will be appreciated that the foregoing description provides examples of the invention. However, it is contemplated that other implementations of the invention may differ in detail from the foregoing examples. All references to the invention or examples thereof are intended to reference the particular example being discussed at that point and are not intended to imply any limitation as to the scope of the invention more generally. All language of distinction and disparagement with respect to certain features is intended to indicate a lack of preference for those features, but not to exclude such from the scope of the invention entirely unless otherwise indicated.

What is claimed is:

1. A needle array for delivering a medicament, the needle array comprising:
an injection needle configured to deliver the medicament to a patient; and
a sensing microneedle configured to sense a characteristic associated with contact between the sensing microneedle and the patient,
wherein a tip of the sensing microneedle is offset from a tip of the injection needle.

2. The needle array of claim 1, wherein the characteristic associated with the contact between the sensing microneedle and the patient is associated with a depth of penetration of the sensing microneedle into the patient.

3. The needle array of claim 2, wherein the depth of penetration is quantified by an amount of contact between the sensing microneedle and the patient along a length of the sensing microneedle, the length being measured between a base of the needle array and the tip of the sensing microneedle.

4. The needle array of claim 1, wherein:
the injection needle and the sensing microneedle are each immovably fixed to a base of the needle array, and
the tip of the injection needle extends distally to or beyond the tip of the sensing microneedle.

5. The needle array of claim 1, wherein the sensing microneedle is a solid microneedle and the injection needle is a hollow needle.

6. The needle array of claim 1, wherein, when the needle array is at a target position on the patient, the tip of the sensing microneedle extends at least through a stratum corneum layer of the patient.

7. The needle array of claim 1, wherein the tip of the sensing microneedle extends less than 1000 μm from a base of the needle array.

8. The needle array of claim 1, wherein the characteristic is at least one of impedance, resistance, or capacitance.

9. The needle array of claim 1, wherein:
the sensing microneedle is one of a plurality of sensing microneedles configured to sense the characteristic,
the plurality of sensing microneedles are arranged in a first sensing microneedle sub-array and a second microneedle sub-array, and
the injection needle is between the first sensing microneedle sub-array and the second microneedle sub-array.

10. The needle array of claim 1, wherein:
the injection needle is a first injection needle and the needle array further comprises a second injection needle configured to also deliver the medicament to the patient, and
the first injection needle and the second injection needle are each hollow microneedles.

11. A wearable medicament delivery device comprising:
a container containing a medicament;
a needle array comprising:
an injection needle configured to deliver the medicament to a patient; and
a sensing microneedle configured to sense a characteristic associated with a depth that the sensing microneedle is inserted into the patient, wherein a tip of the sensing microneedle is offset from a tip of the injection needle;
a pump operatively connected to the injection needle and to the container; and
a controller operatively connected to the pump and the sensing microneedle, the controller being configured to prevent operation of the pump when the characteristic sensed by the sensing microneedle is outside a target range.

12. The wearable medicament delivery device of claim 11, wherein the controller is configured to operate the pump when the characteristic sensed by the sensing microneedle is within the target range.

13. The wearable medicament delivery device of claim 11, wherein the characteristic is impedance.

14. The wearable medicament delivery device of claim 11, further comprising a first housing and a second housing configured to be removably attached together,
wherein the first housing is reusable and houses the controller, and
wherein the second housing is disposable and houses the pump, the container, and the needle array.

15. The wearable medicament delivery device of claim 14, further comprising an adhesive patch attached to the second housing and configured to adhere the wearable medicament delivery device to the patient.

16. The wearable medicament delivery device of claim 11, wherein the container contains more than 0.5 mL of the medicament, and
the controller is configured to control the pump to deliver an entirety of the medicament to the patient via the injection needle.

17. A method of delivering a medicament, the method comprising:
sensing, using a sensing microneedle, a characteristic associated with a depth that the sensing microneedle is inserted into a patient;
determining, using a controller operatively connected to the sensing microneedle, that the characteristic is within a target range; and
switching a state of the controller in response to determining that the characteristic is within the target range from a default state to a ready-to-inject state, wherein in the default state the controller does not operate a pump in response to receiving instructions from a user interface to deliver the medicament, and wherein in the ready-to-inject state the controller operates the pump in response to receiving instructions from the user interface to deliver the medicament.

18. The method of claim 17, further comprising:
determining, using the controller, that an injection has started;
sensing, using the sensing microneedle and during the injection, a second characteristic;
determining, using the controller, that the second characteristic is outside the target range; and
switching, in response to determining that the second characteristic is outside the target range, the state of the controller from the ready-to-inject state to the default state.

19. The method of claim 18, further comprising:
sensing, using the sensing microneedle and after switching the controller to the default state, a third characteristic;
determining, using the controller, that the third characteristic is within the target range; and
switching, in response to determining that the third characteristic is within the target range, the state of the controller from the default state to the ready-to-inject state.

20. The method of claim 17, further comprising:
determining, using the controller, that injection of the medicament has been completed; and
switching, in response to determining that the injection of the medicament has been completed, the state of the controller from the ready-to-inject state to a spent state, in which the controller does not operate the pump in response to receiving instructions from the user interface to deliver the medicament independent from the sensing microneedle.
